(19) United States
(12) Patent Application Publication
(10) Pub. No.: US 2006/0282913 A1
(43) Pub. Date: Dec. 14, 2006

(54) Method of efficiently growing modified morning glory plant

(75) Inventors: Keiichi Shimizu, Kagoshima (JP); Fumio Hashimoto, Kagoshima (JP); Yusukie Sakata, Kagoshima (JP)

Correspondence Address: SQUIRE, SANDERS & DEMPSEY L.L.P., 14TH FLOOR, 8000 TOWERS CRESCENT, TYSONS CORNER, VA, 22182, US

(73) Assignee: SKILL/VISION CO., LTD.

(21) Appl. No.: 10/570,944

(22) PCT Filed: Sep. 10, 2004
(86) PCT No.: PCT/JP04/13206

§ 371 Date: Jun. 21, 2006

(30) Foreign Application Priority Data
Sep. 16, 2003 (JP) 2003-322940

(51) Int. Cl. C12N 15/82; C12N 15/87; A01H 1/00

(52) U.S. Cl. 800/278

(57) ABSTRACT

The present invention provides a method for efficient tissue culturing of modified morning glory; a method for maintaining and proliferating embryoid bodies throughout the year; and a method for obtaining a large number of modified morning glories from embryoid bodies. The present invention was completed by maintaining and proliferating embryoid bodies by tissue culturing of immature embryo obtained from an individual heterozygous for a mutation as modified morning glory; selecting embryoid bodies capable of exclusively producing modified morning glory; and regenerating plant bodies from the embryoid bodies.

### TECHNICAL FIELD

The present invention relates to a method for efficiently proliferating modified morning glory by subjecting explants thereof to tissue culturing. Particularly, the present invention relates to a method conducted in tissue culturing process used in association with a process of breeding, which method comprises clonal proliferation of a certain genotype during breeding, and also relates to regeneration of plant by tissue culturing technique.

### BACKGROUND ART

A modified morning glory is defined as a morning glory having leafs and flowers of altered shapes as a result of mutation, and such modified morning glories with high ornamental value are mostly sterile. A mutation gene that gives higher ornamental value to the modified morning glory is expressed only when the gene is homozygous recessive, as is explained by Mendel's laws. Though an individual heterozygous for the mutation, i.e. individual having a mutation gene and a non-mutation gene on the same locus, does not have phenotype of high ornamental value, it can produce seeds (non-patent document 1, Eiji Nitasaka, Nikkei Science, Vol. 31 No. 9, 70-79, 2001). It is expected that a number of seeds obtained from such a plant will contain a certain ratio of homozygotes for the mutation that will produce modified morning glory of enhanced ornamental value. In this manner, modified morning glory has conventionally been maintained and proliferated by making use of individuals heterozygous for the mutation (non-patent document 1, Eiji Nitasaka, Nikkei Science, Vol. 31 No. 9, 70-79, 2001).

For example, one strain of modified morning glory ‘Aosakaikaryuubaseisaisakibotan’ has two sterility recessive mutations, ‘yanagi (willow-like leaf)’ and ‘botan (peony-like flower)’. Therefore, an individual homozygous for even one of these two recessive mutations is sterile. These sterile individuals are traditionally called ‘demono’. In order to maintain the sterile strain, an individual heterozygous for both recessive mutations should be retained. This heterozygous individual is traditionally called ‘parent tree’. Typically, demono in modified morning glory strain is succeeded in a form of seed from a parent tree which gives 20-30 seeds, and when they are seeded, 4 different phenotypes appear.

Amongst these, an individual homozygous for sterile mutant other than botan is traditionally called “hitoe (single) demono”, and an individual homozygous only for botan mutant is called ‘parent botan’. An individual homozygous for both recessive yanagi and botan mutant produces gorgeous and peculiar flowers, and is traditionally called ‘botan demono’. An individual producing a normal flower falls in the above-mentioned ‘parent tree’.

Phenotypes of parent tree, parent botan, hitoe demono and botan demono follow the Mendel's low, and an expected appearance ratio thereof is 9:3:3:1. Though the phenotypes of parent trees are all the same, genotypes are different. As mentioned above, in order to maintain the strain, the parent tree heterozygous for these two recessive mutations should be retained. To identify such a parent tree, it is necessary to conduct a test seeding and to assay heterozygosity of progenies. Such processes for maintaining and proliferating modified morning glory require a large amount of labor (non-patent document 1, Eiji Nitasaka, Nikkei Science, Vol. 31, No. 9, 70-79, 2001).

As a method for proliferating plant which is hardly proliferated by seed propagation, tissue culturing process has been used. With respect to proliferation of morning glory by tissue culturing, a report has been made in which a plant body was regenerated through an embryoid body from an immature embryo of morning glory (non-patent document 2, Jia and Chua, Plant Sci., 87: 215-223, 1992). It has also been reported that an embryoid body-forming callus was induced and a plant body was regenerated by use of an immature embryo of morning glory (non-patent document 3, Ohtani and Shimada, Plant Biotech., 15: 127-129, 1998). There is another report in which embryoid bodies of morning glory were proliferated by subculturing (non-patent document 4, Shimizu et al., Breeding Research, Vol. 4, Supplementary Vol. 1: 173, 2002).

The term “embryoid body” herein means an embryo-like structure formed from a somatic cell or the like, unlike a regular embryo formed from a zygote resulting from fertilization in a plant body. When clonal proliferation of a plant is schemed, this embryoid body is frequently used. By culturing embryoid bodies, new embryoid bodies can be obtained from old embryoid bodies (non-patent documents 2 and 4).

Conventionally, all typical proliferation methods by tissue culturing of morning glory utilize immature embryo. From an immature embryo of morning glory, an embryoid body or an embryoid body-forming callus is induced, and a plant body is regenerated (non-patent documents 2-6).

There is an report in which a regenerated plant can be obtained by culturing an immature embryo of morning glory in a liquid culture medium and forming an adventive embryo from a callus (non-patent document 5, Shimizu et al., The Journal of Japanese Society for Horticultural Science, Vol. 71, supple mentary Vol. 2: P. 232, 2002). The same content is filed as Japanese patent unexamined application No. 2003-103568 entitled “Method for efficiently proliferating morning glory” (patent document 1, paragraph 0009). In this patent document, there is found a description “studies have been made with respect to a composition of a liquid culture medium for tissue culturing of morning glory, and by selecting a callus having capability of regeneration, the present invention was completed,”. In U.S. Pat. No. 6,649,748 filed on Nov. 11, 2003 (patent document 2) entitled “Peroxidase genomic gene derived from Ipomoea batatas and promoter thereof”, there is found a description with respect to sweet potato, which is a plant of Ipomoea genus to which morning glory also belongs, “The present invention relates to a stress inducible promoter. Particularly, it relates to a promoter of a genomic gene coding a peroxidase isoenzyme of Ipomoea batatas”. In a specification of European patent No.0409320 (patent document 3) entitled “Method for isolating hydroxy fatty acid from Convolvulaceae plant to which morning glory belongs”, there is found a description “the present invention provides a method for isolating a hydroxy fatty acid-related material from Convolvulaceae plant, such as sweet potato”. In either case, tissue culturing of modified morning glory is not mentioned.

[Patent Document 1]

Japanese unexamined patent application No. 2003-103568 publication

[Patent Document 2]

U.S. Pat. No. 6,649,748 specification

[Patent Document 3]

European patent No. 0409320 specification

[Non-Patent Document 1]

Eiji Nitasaka, “Henka Asagao no Rekishi to Idengaku (history and genetics of Japanese morning glory)”, Nikkei Science, 2001, Vol. 31, No. 9, P. 70-79.

[Non-Patent Document 2]

Jia, S. R. and Chua. N. H., “Somatic embryo genesis and plant regeneration from immature embryo culture of Pharbitis nil”, Plant Sci., 1992, Vol. 87, 215-223.

[Non-Patent Document 3]

Otani, M. and Shimada, T., “Embryogenic callus formation from immature embryo of Japanese morning glory (Pharbitis nil Choisy)”, Plant Biotech., 1998, Vol. 15, P. 127-129.

[Non-Patent Document 4]

Keiichi Shimizu et al., “Establishment of efficient transformation of morning glory (Ipomoea nil) using Agrobacterium tumefaciens”, Breeding Research, 2002, Vol. 4, supplementary Vol. 1, P. 173.

[Non-Patent Document 5]

Keiichi Shimizu et al., “Efficient plant regeneration from suspension cultures in Japanese morning glory (Ipomoea nil)”, The Journal of Japanese Society for Horticultural Science, 2002, Vol. 71, supplementary Vol. 2, P. 232.

[Non-Patent Document 6]

Shimizu, K. et al., “Plant Regeneration from Suspension Cultures in Japanese Morning Glory (Ipomoea nil (L.) Roth.)”, J. Japan. Soc. Hort. Sci., 2003, Vol. 72, P. 409-414.

### DISCLOSURE OF INVENTION

Problem to be Solved by the Invention

However, no report has been made with respect to culturing of modified morning glory, since it is impossible to obtain immature embryo from a modified morning glory itself. In addition, no report has been made with respect to regeneration of plant body of modified morning glory from tissue culture. Moreover, it is impossible to proliferate modified morning glory by root division or herbaceous cutting, since morning glory is therophyte. To sum up, no methods have been known for mass proliferation of plant body of modified morning glory.

The present invention provides a method for efficiently conducting tissue culturing of modified morning glory; a method for maintaining and proliferating embryoid bodies throughout the year; and a method for obtaining a large number of modified morning glories. Consequently, the present invention made modified morning glory, which has not been marketed, available to regular consumer.

Means to Solve the Problems

The present inventors made intensive and extensive studies with the view towards solving the above-mentioned problems. As a result, the present inventors found that plant bodies of modified morning glory can be regenerated by: obtaining immature embryos from an individual heterozygous for the mutation as modified morning glory; proliferating embryoid bodies by tissue culturing of the immature embryos; selecting embryoid bodies that give modified morning glory from the proliferated embryoid bodies; and regenerating plant bodies from the selected embryoid bodies, and thus completed the present invention.

The method for efficiently proliferating modified morning glory of the present invention provides a means for proliferating: botan demono that has 2 or more mutations each giving sterility and appears with a ratio of 1/16 or less; parent botan and hitoe demono each of which has one mutation giving sterility and both together appear with a ratio of 1/4; and parent tree.

The method for efficiently proliferating modified morning glory of the present invention includes, as a step of inducing embryoid bodies, a step of separately culturing individual explant obtained from an individual heterozygous for modified morning glory genes and inducing embryoid strains from separate immature embryos, or a step of culturing pieces obtained by cutting flower bud of modified morning glory.

Examples of tissue to be used as explant in the present invention include seed, hypocotyl, cotyledon, cotyledon pedicel, stem, growing point, leaf, petiole, root, petal, sepal, anther, filament, stigma, ovary, ovule, flower bud and immature embryo. Amongst these, immature embryo and flower bud are preferred, since they produce a large number of modified morning glories.

The step of maintaining embryoid bodies may include a step of subjecting individual induced embryoid strain to separate subculturing and then repeatedly and sequentially generating embryoid bodies. It should be noted that, when flower bud of modified morning glory is used, this step can be omitted.

The step of selection may include a step of culturing a part of each embryoid strain, germinating the embryoid bodies, initiating root growth, acclimatizing the grown embryoid bodies, initiating blooming, and selecting a strain that produces modified morning glory from a number of embryoid strains. It should be noted that, when flower bud of modified morning glory is used, this step can be omitted.

The step of proliferating embryoid bodies includes a step of maintaining the process of proliferating the selected embryoid strain and thus proliferating embryoid bodies of modified morning glory throughout the year.

The step of regenerating plant body includes a step of culturing embryoid bodies of modified morning glory selected and proliferated, germinating the embryoid bodies, initiating root growth, acclimatizing the grown embryoid bodies, and producing a large number of plant bodies of modified morning glory.

It is preferred that a medium used in the present invention be mainly composed of a regular basal medium for culturing tissue (e.g. MS, N6, B5 and NN) arbitrary selected, to which sugars as a carbon source and gellan gum or agar as a jellying agent are added.

The method for proliferating modified morning glory of the present invention is characterized in that the method includes a step of subjecting explants of modified morning glory to tissue culturing and inducing embryoid bodies capable of producing modified morning glory; and a step of proliferating such embryoid bodies and regenerating plant bodies from the embryoid bodies. The explant to be used may be flower bud.

In addition, the present invention is characterized in that the method includes a step of subjecting explants of parent tree of modified morning glory to tissue culturing and inducing embryoid bodies capable of producing modified morning glory; a step of selecting such embryoid bodies; and a step of proliferating the selected embryoid bodies and regenerating plant bodies from the embryoid bodies. Further, the present invention is characterized in that the method includes a step of subjecting explants of parent tree of modified morning glory to tissue culturing and inducing embryoid bodies capable of producing modified morning glory or parent trees of modified morning glory; a step of selecting such embryoid bodies; and a step of proliferating the selected embryoid bodies and regenerating plant bodies from the embryoid bodies. The explant to be used may be immature embryo.

The method for proliferating modified morning glory is characterized in that subculturing is introduced to the step of proliferating embryoid bodies and the step of maintaining embryoid bodies. An interval of the subculturing is selected from 1-5 weeks, preferably from 3-4 weeks.

### BRIEF DESCRIPTION OF DRAWINGS

Each of FIG. 1A and FIG. 1B shows a flow chart from obtaining explant of modified morning glory to regenerating plant body.

FIG. 2 shows culturing of immature embryos of modified morning glory and formation and maintenance of embryoid bodies.

FIG. 3 shows a method for selecting embryoid bodies capable of regenerating modified morning glory.

FIG. 4 shows proliferation of the selected embryoid bodies and regeneration of modified morning glory from the embryoid bodies.

FIG. 5 shows a comparison between culturing of immature embryo in a liquid culture media and culturing on a solid medium.

### BEST MODES FOR CARRYING OUT THE INVENTION

Modes for carrying out the present invention will be described in detail below.

Morning glory to be used for the method of the present invention is a generic name for plants having a scientific name Ipomoea nil which belongs to Convolvulaceae family, or for crossbreed plants of Ipomoea nil with other plant species, and may include those wildly inhabiting Japan, other Asian countries, Africa and South America. The term “modified morning glory” in the present invention indicates morning glory that has leafs or flowers of unusual shapes as a result of mutation, and thus looks remarkably different from regular morning glory.

Examples of the modified morning glory in the present invention include, but are not specifically limited to,

| | | | | | | Flower | | | | | | |
|---|---|---|---|---|---|---|---|---|---|---|---|---|
| Leaf | | | | | | | | | | Petal | | |
| | | | For- | | | | | Tube | | Appear- | | |
| Color | Texture | Sex | mation | Type | Stem | Color | Pattern | Color | Petal | ance | How to Flower | Double |
| Blue | | | | Sukuitsu | | Lapis | | | Souhuurin | | Shishizaki | Peony |
| | | | | meryuuba | | | | | | | | |
| Blue | | | | Onogatu | | White | | | Soukanben | Metepr | Shishizaki | Peony |
| | | | | meryuuba | | | | | | | | |
| Blue | | | | Sukuitsu | | Red | marginal | | Soutarikau | | Shishizaki | Peony |
| | | | | meryuuba | | | variegation | | | | | |
| yellow | | | | Nlelritsu | | Red Purple | | | Wind Bell | | Kamllrishishizaki | Peony |
| | | | | meryuuba | | | | | | | | |
| yellow | | | Shrink | Tatsutasi | | Dark Blue | | | Soutarikau | | Fukiagekurumazaki | Peony |
| | | | | [symbol] | | Green | | | | | | |
| yellow | | | Shrink | Tatsutak | | Pale Purple | | | | | Fukiagekurumazaki | Peony |
| | | | | arakueaa | | Gray | | | | | | |
| | | | | meryuuba | | | | | | | | |
| yellow | | | Shrink | Sasomery | | Deep | | | | | Suzumesaki | Peony |
| | | | | [symbol] | | Purple | | | | | | |
| | | | | | | Gray | | | | | | |
| Blue | | | Idaki | Leaf | | Bluish | | | | | Doizaki | Peony |
| | | | Shrink | | | White | | | | | | |
| Blue | Quartz | Eddy | | Willow | | Dark Lilac | Nail | | | Saizaki | Bottom | |
| | spot | | | Leaf | | | marginal | | | | | |
| | | | | | | | variegation | | | | | |
| Blue | | Eddy | | Willow | | Edomunisaki | | | | | Saizaki | Peony |
| | | | | Leaf | | | | | | | | |
| Blue | | Bukko | | Willow | | Blue | | | | | Saizaki | Peony |
| | | mikai | | Leaf | | | | | | | | |
| | | Eddy | | | | | | | | | | |
| blue | | Bukko | | Willow | | White | | | | | Saizaki | Peony |
| | | mikai | | Leaf | | | | | | | | |
| | | Eddy | | | | | | | | | | |
| yellow | | Bukko | | Willow | | Blue Purple | Hubuki | | | | Nadeshikceaizaki | Peony |
| | | mikai | | Leaf | | on Light | | | | | | |
| | | Eddy | | | | Blue | | | | | | |
| blue | | Eddy | | Kuwagata | | Lilac | | | | | Nadeshikceaizaki | |
| | | | | willow | | Purple | | | | | | |
| | | | | Leaf | | | | | | | | |
| blue | | | Shrink | Round | | Purple | Nail | | | | Saizaki | |
| | | | | Willow | | | marginal | | | | | |
| | | | | Leaf | | | variegation | | | | | |
| blue | | Bukko | | Kagerou | | Blue | | | | | Nadeshikceaizaki | Peony |
| | | mikai | | Willow | | | | | | | | |
| | | Eddy | | Leaf | | | | | | | | |
| blue | | Eddy | | Kuwagata | | Lilac | | | | | Nadeshikceaizaki | Peony |
| | | | | willow | | Purple | | | | | | |
| | | | | leaf | | | | | | | | |
| yellow | | Eddy | | Willow | Hung | Blue | | | | | Nadeshikceaizaki | Peony |
| | | | | Leaf | Branch | | | | | | | |
| blue | | | | Kakae | | Purple | | | | | Nadeshikceaizaki | Peony |
| | | | | Willow | | | | | | | | |
| | | | | Leaf | | | | | | | | |
| blue | | | | Shishi | | Lapis | | | | | Saizaki | Peony |
| | | | | Willow | | | | | | | | |
| | | | | Leaf | | | | | | | | |
| blue | | | | Willow | | Lilac | | | | | Nadeshikceaizaki | Peony |
| | | | | Leaf | | Purple | | | | | | |
| yellow | | Eddy | | Willow | | Tsubame | | | | | Nadeshikceaizaki | |
| | | | | Leaf | | Purple | | | | | | |
| blue | | | | Fine | | Red | | | | | Saizaki | |
| | | | | Willow | | | | | | | | |
| | | | | Leaf | | | | | | | | |
| blue | | | | Thread | | Lilac | | | Fine | | Saizaki | Peony |
| | | | | Willow | | Purple | | | | | | |
| | | | | Leaf | | | | | | | | |
| blue | | | | Sea blue | | Red | | | | | Saizaki | Peony |
| | | | | Leaf | | | | | | | | |
| blue | | | | Rinpou | | White | | | | | Saizaki | Peony |
| | | | | thread | | | | | | | | |
| | | | | willow | | | | | | | | |
| | | | | Leaf | | | | | | | | |
| blue | | | Shrink | Thread | | Lilac | Kurumashi- | | Fine | | Saizaki | Peony |
| | | | | Willow | | Purple | bori | | | | | |
| | | | | Leaf | | | | | | | | |
| blue | | | | Thread | | Lilac | | | Fine | | Saizaki | Peony |
| | | | | Willow | | Purple | | | | | | |
| | | | | Leaf | | | | | | | | |
| blue | | | | Thread | | Lilac | | | Fine | | Saizaki | Peony |
| | | | | Willow | | Purple | | | | | | |
| | | | | Leaf | | | | | | | | |
| blue | | | | Thread | | Very Pale | | | Fine | | Saizaki | Peony |
| | | | | Willow | | Purple | | | | | | |
| | | | | Leaf | | | | | | | | |
| blue | | | | Thread | | Very Pale | | | Fine | | Saizaki | Peony |
| | | | | Willow | | Purple | | | | | | |
| | | | | Leaf | | | | | | | | |
| blue | | | | Thread | | Very Pale | | | Fine | | Saizaki | Peony |
| | | | | Willow | | Purple | | | | | | |
| | | | | Leaf | | | | | | | | |
| blue | | | | Thread | | Lilac | | | Fine | | Saizaki | Peony |
| | | | | Willow | | Purple | | | | | | |
| | | | | Leaf | | | | | | | | |
| blue | | | | Thread | | Lilac | | | Fine | | Saizaki | Peony |
| | | | | Willow | | Purple | | | | | | |
| | | | | Leaf | | | | | | | | |
| blue | | | | Thread | | Very Pale | | | Fine | | Saizaki | Peony |
| | | | | Willow | | Purple | | | | | | |
| | | | | Leaf | | | | | | | | |
| blue | Spots | | | Thread | | Lilac | | | Fine | | Saizaki | Peony |
| | | | | Willow | | Purple | | | | | | |
| | | | | Leaf | | | | | | | | |
| blue | | | | Willow | | Pale Pink | | | | | Saizaki | Peony |
| | | | | Leaf | | | | | | | | |
| yellow | | | | Willow | | Lapis | | | | | Saizaki | Peony |
| | | | | Leaf | | | | | | | | |
| blue | | | | Willow | | Blue Purple | | | Fine | | Saizaki | Peony |
| | | | | Leaf | | | | | | | | |

Examples of the morning glory to be used as a parent tree of modified morning glory include, but are not restricted to, plants having a scientific name of Ipomoea nil which belongs to Convolvulaceae family, and crossbreed plants of Ipomoea nil with other plant species, including those wildly inhabiting Japan, other Asian countries, Africa and South America.

The embodiments for carrying out the present invention will be described below with reference to the accompanying drawings.

<Step of Inducing Embryoid Bodies>

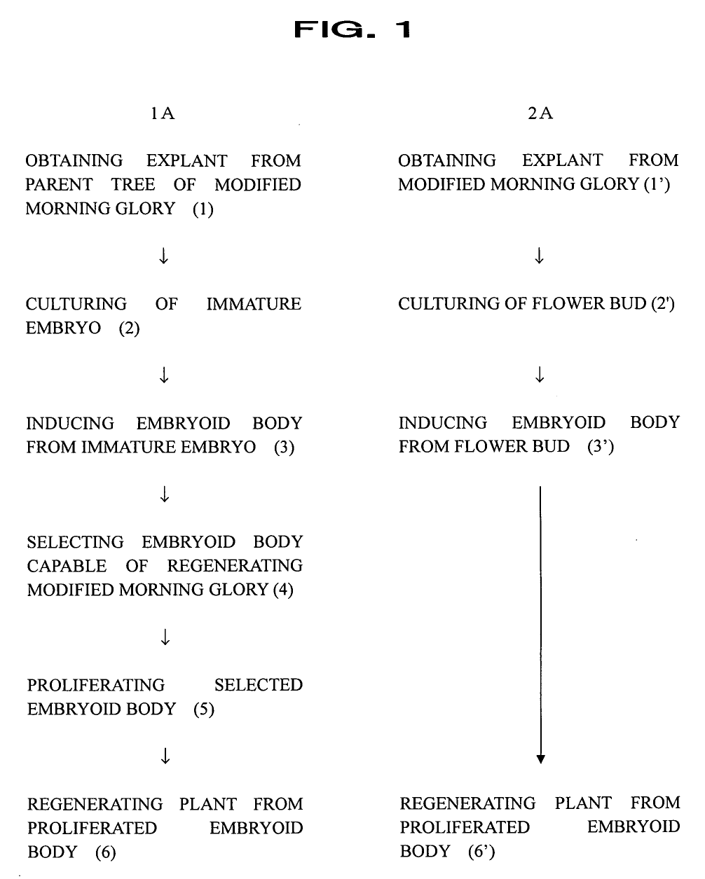

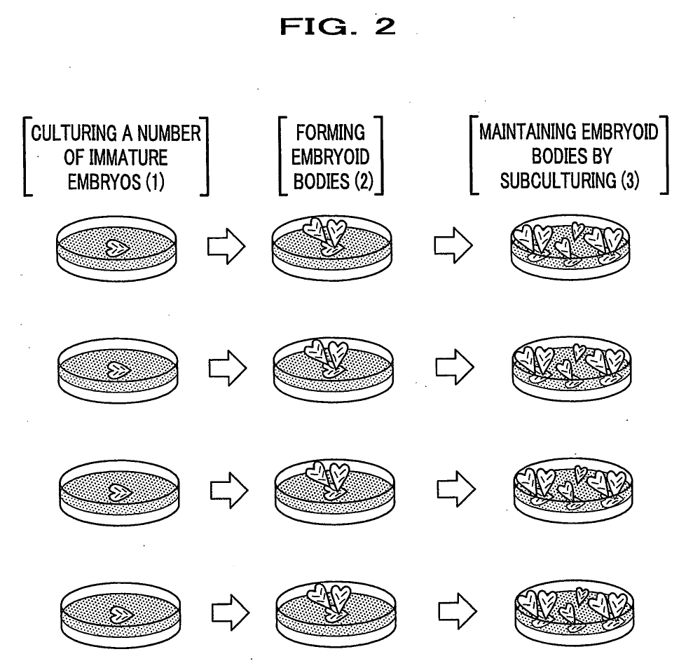

Referring to (1)-(2) of FIG. 1A and (1) of FIG. 2, first, immature embryos of a morning glory heterozygous for mutation as modified morning glory are obtained. Subsequently, each individual immature embryo is separately cultured in a medium containing NAA. After culturing the immature embryo for 3-4 weeks, embryoid bodies are formed as shown in (2) of FIG. 2.

Examples of the tissue to be used as explant in the step for inducing embryoid bodies include seed, hypocotyl, cotyledon, cotyledon pedicel, stem, growing point, leaf, petiole, root, petal, sepal, anther, filament, stigma, ovary, ovule, flower bud and immature embryo. The tissue is cultured in a predetermined medium, such as a medium containing NAA. The tissue is preferably immature embryo or flower bud, which produces a large number of modified morning glories.

The immature embryo is cultured on a medium containing NAA. A medium concentration of NAA suitable for culturing is 1-9 mg/l, preferably 2-4 mg/l. For this medium, a regular basal medium for tissue culturing, such as MS, N6, B5 and NN media, arbitrary selected can be used, and an N6 medium is especially preferred. In this medium, sugars, such as sucrose, fructose, glucose and trehalose, can be used as a carbon source, and amongst these, sucrose is especially preferred. For a medium concentration of the sugar, 10-90 g/l is desirable, and 60 g/l is especially preferred. For a jellying agent to be used as a support in the medium, jellying agents conventionally used in this field can be used, such as agar and gellan gum. Amongst these, gellan gum is more preferred. For a medium concentration of the jellying agent, in the case of gellan gum for example, 0.2-5 g/l is desirable, and 3-4 g/l is especially preferred. For a temperature condition of culturing, 20-30° C. is desirable, and 24-26° C. is especially preferred. It should be noted that the culturing is conducted under light irradiation, such as light irradiation of 100-8000 lux.

<Step of Maintaining Embryoid Bodies>

The embryoid bodies obtained as shown in (3) of FIG. 1A and (3) of FIG. 2 are subcultured on a medium having the same composition as the medium mentioned above. By making use of subculture, new embryoid bodies are generated from the old embryoid bodies, and thus embryoid bodies are maintained. During subculturing, an embryoid body derived from different immature embryo is treated separately as a discrete strain.

In the step of maintaining embryoid bodies, the obtained embryoid bodies are subcultured on the same medium as the medium used for inducing embryoid bodies. As a result, new embryoid bodies are generated from the old embryoid bodies, and thus embryoid bodies can be maintained.

<Step of Selecting Embryoid Bodies>

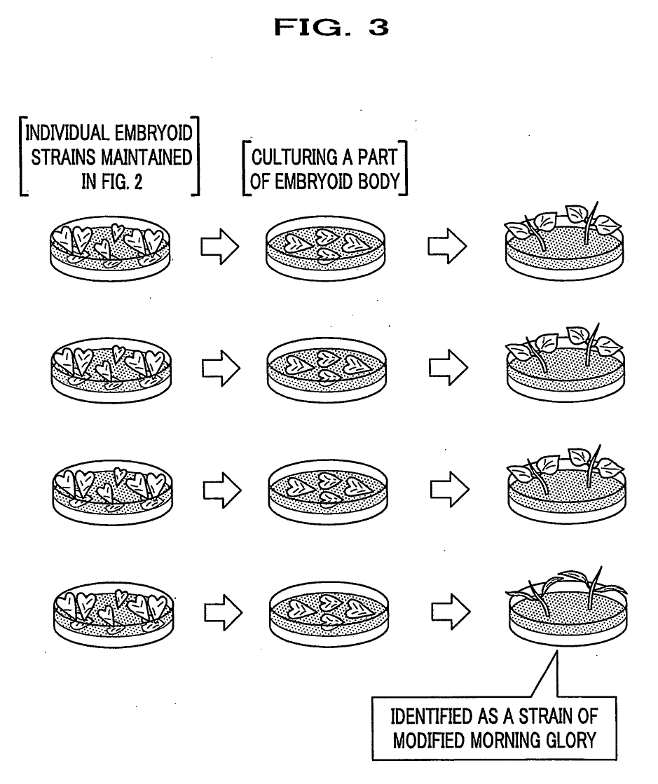

From the embryoid strains maintained as shown in (4) of FIG. 1A and FIG. 3, an embryoid strain capable of regenerating modified morning glory is selected. From each strain, a part of embryoid body is obtained. The obtained embryoid body is cultured in a medium containing auxin and cytokinin, and a shoot is germinated. On a medium containing no phytohormone, the germinated shoot is cultured and root growth is initiated, to thereby regenerate plant body and initiate blooming. Then, only an embryoid strain is selected which produced embryoid bodies that ultimately bloomed as modified morning glory.

In the step of selecting embryoid bodies, a shoot was formed from the embryoid body. As for a medium to be used for forming the shoot from the embryoid body, a conventional medium to be used for tissue culturing, such as MS, N6, B5 and NN media, can be used. The shoot may be formed regardless of a presence of phytohormone in the medium. It is desirable that a blend of auxin and cytokinin be added to the medium, and specifically, 0.1-0.5 mg/l of indoleacetic acid and 1-2 mg/l of benzylaminopurine be added to the medium. A type and a concentration of sugar to be used in the medium are the same as those used for typical tissue culturing, and for example, approximately 25-35 g/l of sucrose can be added. Temperature and light conditions for culturing are the same as those used for the embryoid formation. For a jellying agent as a support of the medium, either agar or gellan gum may be used, though agar is more preferred. It is preferred that a medium concentration of the jellying agent be 8-15 g/l, in the case of agar, for example.

<Step of Proliferating Embryoid Bodies>

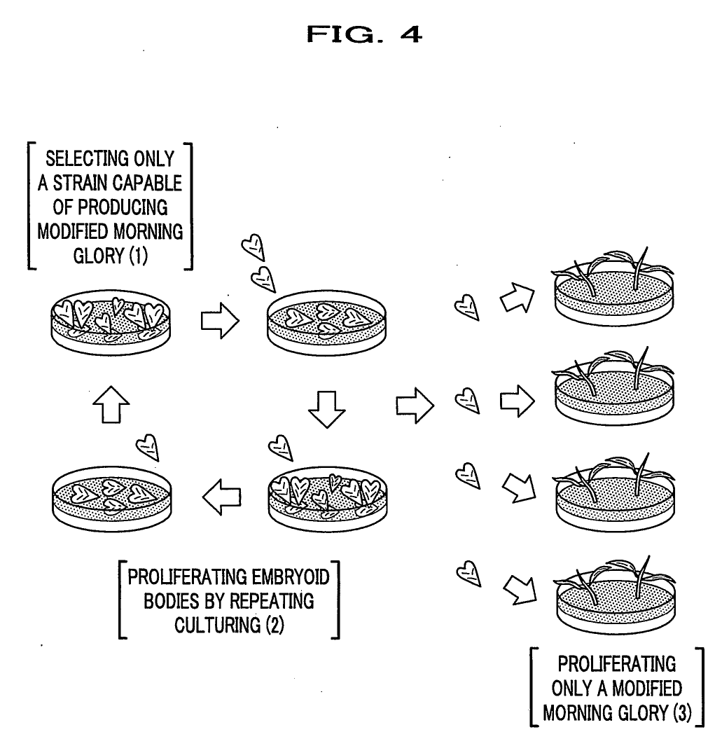

As shown in (5) of FIG. 1A and (2) of FIG. 4, the selected embryoid strain of modified morning glory is subcultured on a medium having the same composition as the medium described above. By making use of subculture, new embryoid bodies are generated from old embryoid bodies. By repeating subculturing at intervals of 1-4 weeks, a large number of embryoid bodies are formed.

In the step of proliferating embryoid bodies, the obtained embryoid bodies are cultured on the same medium as the medium used for inducing embryoid bodies. As a result, new embryoid bodies are generated from the old embryoid bodies, and thus embryoid bodies can be proliferated. The proliferation of embryoid bodies was further continued by repeating subculturing. One subculturing is conducted for 1-5 weeks, preferably 3-4 weeks. Experiments by the present inventors demonstrated that an ability to form embryoid body was maintained after repeating subculturing for more than one year.

<Step of Producing Modified Morning Glory>

As shown in (6) of FIG. 1A and (3) of FIG. 4, a part of embryoid bodies proliferated in a large amount is cultured in a medium containing auxin and cytokinin, and a shoot is germinated. On a medium containing no phytohormone, the germinated shoot is cultured and root growth is initiated, to thereby regenerate plant body and thus a number of modified morning glories can be proliferated.

In the step of producing modified morning glory, a shoot was formed from the embryoid body. As for a medium to be used for forming the shoot from embryoid body, a conventional medium to be used for tissue culturing, such as MS, MS, N6, B5 and NN media, can be used,. The shoot may be formed regardless of a presence of phytohormone in the medium. It is desirable that a blend of auxin and cytokinin be added to the medium, and specifically, 0.1-0.5 mg/l of indoleacetic acid and 1-2 mg/l of benzylaminopurine be added to the medium. A type and a concentration of sugar to be used in the medium are the same as those used for typical tissue culturing, and for example, approximately 25-35 g/l of sucrose can be added. Temperature and light conditions for culturing are the same as those used for the embryoid formation. For a jellying agent as a support of the medium, either agar or gellan gum may be used, though agar is more preferred. It is preferred that a medium concentration of the jellying agent be 8-15 g/l, in the case of agar, for example.

In order to initiate root growth in the shoot formed above, the shoot is cultured on a regular medium for culturing tissue, such as an MS medium, arbitrary selected and seedling plant was regenerated. A type and a concentration of sugar to be used in the medium are the same as those used for typical tissue culturing, and for example, approximately 30 g/l of sucrose can be added. Temperature and light conditions for culturing are the same as those used for the embryoid formation. For a support of the medium, for example, agar, gellan gum, vermiculite, rock wool or the like can be used.

It should be noted that, in the case of culturing flower bud as shown in (2′) of FIG. 1B instead of culturing immature embryos as shown in (2) of FIG. 1A, embryoid bodies can be induced from a cultured flower bud ((3′) of FIG. 1B), and directly proliferated, and then modified morning glory can be obtained from the proliferated embryoid bodies ((6′) of FIG. 1B). In other words, a step (4) of screening embryoid bodies and a step (5) of proliferating embryoid bodies shown in FIG. 1A can be omitted.

### EXAMPLES

The present invention will be explained below with reference to the following specific Examples. However, the present invention should not be limited by the Examples.

### Example 1A

Immature embryos were obtained from a parent tree of modified morning glory “aosakaikaryuubaseisaisakibotan”. Each individual immature embryo was separately cultured on an MS medium containing 3 mg/l of NAA and 60 g/l of sucrose at 25° C. under 2000 lux, to thereby obtain an embryoid body from the immature embryo. Each embryoid body obtained from different immature embryo was separately cultured as a discrete strain. Subculturing was repeated using the same medium at an interval of 4 weeks.

Next, from a number of embryoid strains subcultured, strains that regenerated modified morning glory were selected. For each strain, a part of the embryoid bodies that had been induced from the immature embryo and kept in subculturing was cultured on an MS medium containing 0.2 mg/l of indoleacetic acid, 2 mg/l of benzylaminopurine, 30 g/l of sucrose and 10 g/l of agar. On this medium, a shoot was formed from the embryoid body. Then, the formed shoot was transferred to an MS medium containing no phytohormone. A plant body with initiated root growth was obtained, acclimatized and bloomed. Embryoid strains that exclusively regenerated modified morning glory were selected. In this case of aosakaikaryuubaseisaisakibotan, 237 immature embryos were cultured, and 11 amongst these were found to be objective homozygotes for Saisaki and Botan.

### Example 1B

Under substantially the same conditions as in Example 1, culturing was conducted using seed, hypocotyl, cotyledon, cotyledon pedicel, stem, growing point, leaf, petiole, root, petal, sepal, anther, filament, stigma and ovary, and the results were compared with the case of culturing of immature embryo. As is apparent from a result shown in Table 1, embryoid bodies were produced solely in the case of culturing immature embryo.

TABLE 1

| | Number | Number of explant |
|---|---|---|
| | of explant | that formed |
| Explant type | cultured | embryoid body |
| Immature | 281 | 221 |
| embryo | | |
| Seed | 10 | 0 |
| Hypocotyl | 11 | 0 |
| Cotyledon | 20 | 0 |
| Cotyledon | 30 | 0 |
| Pedicel | | |
| Stem | 15 | 0 |
| Growing point | 10 | 0 |
| Leaf | 50 | 0 |
| Petiole | 72 | 0 |
| Root | 40 | 0 |
| Petal | 15 | 0 |
| Sepal | 30 | 0 |
| Anther | 23 | 0 |
| Filament | 10 | 0 |
| Stigma | 5 | 0 |
| Ovary | 5 | 0 |

### Example 1C

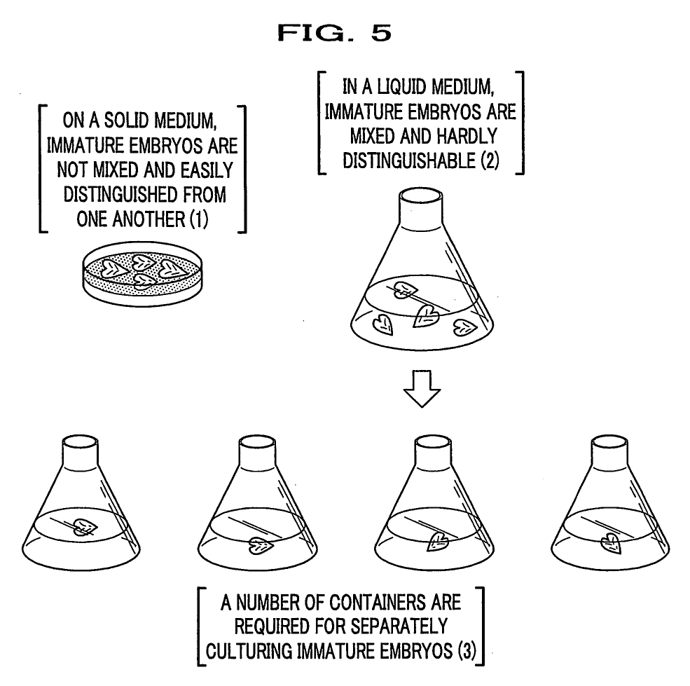

Referring to FIG. 5, comparison is made between a case where modified morning glory is cultured in a liquid culture medium and a case where modified morning glory is cultured on a solid medium. As shown in (2) of FIG. 5, in the case of liquid culturing, immature embryos are mixed in the liquid and it became extremely difficult to distinguish individual immature embryos. Therefore, as shown in (3) of FIG. 5, in order to make distinction between individual immature embryos during culturing, separate container should be prepared for each immature embryos, which requires complicated and time-consuming procedures and a wider culturing space. Contrary, as shown in (1) of FIG. 5, in the case of solid medium, immature embryos are not mixed unlike the case of liquid culture medium. Since a number of immature embryos can be distinguishably cultured in one culturing container, less complicated procedure and smaller space is required as compared with the case of the liquid culture medium.

### Example 1D

In substantially the same manner as in Example 1A, immature embryos were cultured except that phytohormone other than NAA, such as 4-fluorophenoxyacetic acid, 2,4-dichlorophenoxyacetic acid and picloram were used. As is apparent from a result shown in Table 4, in the case of phytohormone other than NAA, no embryoid bodies were produced, and thus mass proliferation was not attained.

TABLE 2

| | Number of embryoid bodies |
|---|---|
| Phytohormone | formed per immature embryo |
| NAA | 10 |
| 4-fluorophenoxyacetic | 0 |
| acid | |
| 2,4-dichlorophenoxyacetic | 0 |
| acid | |
| Picloram | 0 |

### Example 1E

Substantially the same procedures were repeated as in Example 1A, except that the interval for subculturing embryoid bodies of modified morning glory was set 1, 2, 3, 4 and 5 weeks, and results were compared. As is apparent from a result shown in Table 2, the intervals of 3 weeks and 4 weeks gave larger numbers of embryoid bodies. The embryoid bodies were proliferated 7-16 times in a month.

TABLE 3

| | Number of embryoid |
|---|---|
| Interval between | bodies formed after |
| subculturing | 2nd subculturing |
| 1 week | 10 |
| 2 weeks | 15 |
| 3 weeks | 16 |
| 4 weeks | 14 |
| 5 weeks | 7 |
| 6 weeks | 3 |
| 7 weeks | 4 |

### Example 1A

A flower bud having a length of approximately 1 cm was obtained from a modified morning glory. The flower bud was cut into explants of 1-2 mm. Six hundred forty-one explants were cultured on an MS medium containing 3 mg/l of NAA, 3.2 g/l of gellan gum and 60 g/l of sucrose. As a result, one explant formed an embryoid body. The embryoid body was proliferated by subculturing in the same manner as in the case of embryoid body derived from immature embryo. It was found that the resultant proliferated embryoid bodies can be handled in the same manner as in the case of embryoid bodies derived from immature embryo.

### Example 2B

In Example 2A, when subculturing of embryoid bodies was repeated at an interval of 4 weeks, formation of embryoid bodies was maintained for more than 1 year. In contrast, when subculturing was repeated at an interval of 5 weeks or more, no embryoid bodies was formed after 5th subculturing as shown in Table 3.

TABLE 4

| | Number of embryoid |
|---|---|
| Interval between | bodies formed after |
| subculturing | 5th subculturing |
| 4 weeks | 14 |
| 5 weeks | 0 |
| 6 weeks | 0 |
| 7 weeks | 0 |

### Example 3

Embryoid bodies obtained in Examples 1A and 2A that produced modified morning glory were cultured on an MS medium containing 0.2 mg/l of indoleacetic acid, 2 mg/l of benzylaminopurine, 30 g/l of sucrose and 10 g/l of agar, to thereby forming a shoot from each of the embryoid bodies. After transferring the shoots to an MS medium containing no phytohormone, plant bodies with initiated root growth were obtained. The shoot formation and the root growth initiation were conducted at 25° C. under 200 lux. As a result, it was shown that a large number of modified morning glories can be produced.

### Example 4

In substantially the same manner as in Examples 1A and 3, eighty-six immature embryos were obtained from a parent tree of modified morning glory “aosakaikaryuubaseisaisakibotan” and cultured. Three immature embryos amongst these were found to be objective homozygote for both Kassaki and Botan. By subculturing embryoid bodies derived from these immature embryos, the embryoid bodies proliferated 7-17 times in one month, and thus it was shown that a large number of aosakaikaryuubaseisaisakibotan can be produced.

### Example 5

In substantially the same manner as in Examples 1A and 3, sixteen immature embryos were obtained from a parent tree of modified morning glory “Ousyokusyukumentatutakurumasakibotan” and cultured. One amongst these was found to be objective homozygote for both Tatuta and Botan. By subculturing an embryoid body derived from this immature embryo, the embryoid bodies proliferated more than twice, and thus it was shown that a large number of Ousyokusyukumentatutakurumasakibotan can be produced.

=0053=

Though the present invention is explained with reference to the particular embodiments and Examples, the present invention should not be limited to these embodiments and Examples, and may be carried out in various forms, as in items explained below.

(1) A method for proliferating modified morning glory mainly comprising: a step of inducing embryoid body capable of producing modified morning glory from explant by tissue culturing; a step of maintaining embryoid bodies; a step of screening embryoid bodies; a step of proliferating embryoid bodies; and a step of producing modified morning glory.

(2) The method according to item (1) wherein the step of maintaining embryoid bodies and the step of proliferating embryoid bodies comprise subculturing.

(3) The method according to item (1) or (2) wherein the explant is immature embryo.

(4) The method according to any one of items (1)-(3) wherein the subculturing was repeated at an interval selected from 1-5 weeks, preferably 3-4 weeks.

### INDUSTRIAL APPLICABILITY

As explained above, according to the present invention, it becomes possible to establish a method for efficiently conducting tissue culturing of modified morning glory; a method for maintaining and proliferating embryoid bodies throughout the year; and a method for obtaining a large number of modified morning glories.

1. A method for proliferating modified morning glory comprising:
a step of subjecting an explant of modified morning glory to tissue culturing and inducing an embryoid body capable of producing modified morning glory; and
a step of maintaining and proliferating the induced embryoid body and regenerating a plant body.

2. The method according to claim 1, wherein the explant is flower bud.

3. A method for proliferating modified morning glory comprising:
a step of subjecting an explant of a parent tree of modified morning glory to tissue culturing and inducing an embryoid body capable of producing modified morning glory;
a step of screening the induced embryoid body; and
a step of maintaining and proliferating the screened embryoid body and regenerating a plant body.

4. A method for proliferating modified morning glory comprising:
a step of subjecting an explant of a parent tree of modified morning glory to tissue culturing and inducing an embryoid body capable of producing modified morning glory or parent tree of modified morning glory;
a step of screening the induced embryoid body; and
a step of proliferating the screened embryoid body and regenerating a plant body.

5. The method according to claim 3 or 4, wherein the explant is immature embryo.

6. The method according to any one of claims 1, 3 and 4, wherein the step of maintaining and proliferating the embryoid body comprises subculturing.

7. The method according to claim 1, 3 or 4, wherein the subculturing was repeated at an interval selected from 1-5 weeks, preferably from 3-4 weeks.
